(19) United States
(12) Patent Application Publication
(10) Pub. No.: US 2022/0296221 A1
(43) Pub. Date: Sep. 22, 2022

(54) Device and Method for Pre-Term Birth Risk Assessment

(71) Applicant: ECOLE POLYTECHNIQUE FEDERALE DE LAUSANNE (EPFL), Lausanne (CH)

(72) Inventors: Erick Garcia Cordero, Lausanne (CH); Carlotta Guiducci, Lonay (CH); Gloria Porro, Ecublens (CH); David Baud, Lausanne (CH); Loulia Kasem, Ecublens (CH)

(21) Appl. No.: 17/642,274

(22) PCT Filed: Sep. 22, 2020
(86) PCT No.: PCT/IB2020/058817

§ 371 Date: Mar. 11, 2022

(30) Foreign Application Priority Data
Sep. 22, 2019 (IB) PCT/IB2019/058009

(51) Int. Cl. A61B 10/00; B01L 3/00; A61B 5/00; A61F 13/472; G01N 33/68

(52) U.S. Cl. CPC A61B 10/0045; B01L 3/502715; A61B 5/4294; A61F 13/472; A61B 5/7275; G01N 33/689; B01L 2200/027; B01L 2300/0645; B01L 2400/0406; A61F 2013/8473

(57) ABSTRACT

The invention relates to a vaginal fluid monitoring device embedded into a feminine sanitary pad, said device comprising a stack of the following elements: An absorbent layer (100) configured to be in proximity to, and collect, a vaginal fluid; and A biosensing system (1000) in fluidic connection with said absorbent layer (100), said biosensing system (1000) comprising: A microfluidic chip (200) configured to perform an immunoassay to detect the presence and/or the concentration of at least one target biomarker comprised in a vaginal fluid; Means (300) for providing a readout of the presence and/or the concentration of said at least one target biomarker; and An electrode array (400) located along the microfluidic chip (200) configured to detect and analyze the flow of said vaginal fluid by impedance means; wherein said immunoassay is configured to detect the presence and/or the concentration of at least one target biomarker indicative of a pre-term birth (PTB) risk and/or premature rupture of membrane (PROM) risk. The invention also relates to a method for detecting at least one target biomarker comprised in a vaginal fluid.

### TECHNICAL FIELD

The invention lies in the field of medical devices and methods for non-invasively analyzing vaginal secretions for determining a pre-term birth risk.

### BACKGROUND ART

Preterm birth (PTB) risk assessment is a major issue in obstetrics and gynecology. Premature labor onset affects, in fact, one in ten pregnancies and, most importantly, around 50% of the cases are completely unexpected. Preterm labor can originate from several factors. Increasing average age of pregnant women is contributing to the currently observed rise in preterm deliveries: vascular issues are more common with increased age, leading to reduced placenta blood supply; the use of fertility treatments is correlated with multiple gestations, case for which preterm labor is more likely. Among other factors, in several cases preterm birth has been linked to Premature Rupture of Membranes (PROM), i.e. the preterm breakage of the amniotic sac.

Today, preterm birth diagnostics is only performed at the hospital. The patient undergoes a clinical evaluation, and successively, if the doctor notices the presence of symptoms or risk indicators, his diagnosis can be verified performing tests on biomarkers whose concentration in vaginal secretions is directly indicative of preterm birth. The biological sample is collected through an invasive vaginal swab and the testing procedure is conducted by qualified personnel. In case the test is positive, the patient is hospitalized for routine monitoring, leading to discomfort and high costs. Furthermore, no screening procedure is carried out nowadays on asymptomatic women.

As of today, there is no point-of-care, non-invasive, alternative solution to the current standard diagnostic methods for preterm delivery risk assessment.

### SUMMARY OF INVENTION

In order to address and overcome at least some of the above-mentioned drawbacks of the prior art solutions, the present inventors developed a first in class device for assessing the preterm delivery risk.

The purpose of the present invention is therefore that of providing a novel, alternative solution to standard PTB risk diagnostic methods that overcomes or at least reduces the above-summarized drawbacks affecting known solutions according to the prior art.

In particular, a first purpose of the present invention is that of providing a non-invasive, frequent at-home monitoring system for preterm delivery risk assessment.

A further purpose of the present invention is that of providing a comfortable and wearable device matching the requirement of portable bio-analytics with small sample amount.

Still a further purpose of the present invention is that of providing a robust, reliable and user-friendly method for determining a pre-term birth risk through a non-invasive analysis of vaginal secretions.

All those aims have been accomplished with the present invention, as described herein and in the appended claims.

In view of the above-summarized drawbacks and/or problems affecting the solutions of the prior art, according to the present invention there is provided a vaginal fluid monitoring device embedded into a feminine sanitary pad according to claim 1.

Another object of the present invention relates to a method for detecting at least one target biomarker comprised in a vaginal fluid according to claim 11.

Further embodiments of the present invention are defined by the appended claims.

The above and other objects, features and advantages of the herein presented subject-matter will become more apparent from a study of the following description with reference to the attached figures showing some preferred aspects of said subject-matter.

### BRIEF DESCRIPTION OF DRAWINGS

FIG. 1 depicts an exploded view of one embodiment of the vaginal fluid monitoring device embedded into a feminine sanitary pad according to the invention;

FIG. 2 depicts a top view of one embodiment of a microfluidic chip coupled with an electrode array for measuring a vaginal fluid flow through impedance means. For the sake of clarity, the microfluidic chip and the electrode array are shown side by side instead of stacked one on another;

FIG. 3 shows five different embodiments of the fluidic channels and collection areas of an absorbent layer according to the invention. The dotted boxes comprise the five collection areas and half of the fluidic channels of every absorbent layers;

FIG. 4 shows the result of a lateral flow assay for fetal Fibronectin embedded in the paper collection pad. Notice the two lines in the dotted boxes on the colorimetric test strip giving a positive result to the analysis.

### DETAILED DESCRIPTION OF THE INVENTION

The subject-matter herein described will be clarified in the following by means of the following description of those aspects which are depicted in the drawings. It is however to be understood that the subject matter described in this specification is not limited to the aspects described in the following and depicted in the drawings; to the contrary, the scope of the subject-matter herein described is defined by the claims. Moreover, it is to be understood that the specific conditions or parameters described and/or shown in the following are not limiting of the subject-matter herein described, and that the terminology used herein is for the purpose of describing particular aspects by way of example only and is not intended to be limiting.

Unless otherwise defined, technical and scientific terms used herein have the same meaning as commonly understood by one of ordinary skill in the art to which this invention belongs. Further, unless otherwise required by the context, singular terms shall include pluralities and plural terms shall include the singular. The methods and techniques of the present disclosure are generally performed according to conventional methods well known in the art and as described in various general and more specific references that are cited and discussed throughout the present specification unless otherwise indicated. Further, for the sake of clarity, the use of the term “about” is herein intended to encompass a variation of +/−10% of a given value.

The following description will be better understood by means of the following definitions.

As used in the following and in the appended claims, the singular forms “a”, “an” and “the” include plural referents unless the context clearly dictates otherwise. Also, the use of “or” means “and/or” unless stated otherwise. Similarly, “comprise”, “comprises”, “comprising”, “include”, “includes” and “including” are interchangeable and not intended to be limiting. It is to be further understood that where for the description of various embodiments use is made of the term “comprising”, those skilled in the art will understand that in some specific instances, an embodiment can be alternatively described using language “consisting essentially of” or “consisting of.”

For “vaginal fluid”, “vaginal discharge” or “vaginal secretion” is herein meant a mixture of liquid, cells, and bacteria that lubricates and protects the vagina. This mixture is constantly produced by the cells of the vagina and cervix and it exits the body through the vaginal opening. The composition, amount, and quality of discharge varies between individuals as well as through the various stages of sexual and reproductive development. Vaginal fluid secretions are normally produced by women in physiological conditions. The fluid is primarily composed of mucus from cervix and vaginal transudate, whose function is to provide a physiological moisture. Moreover it can contain exudates from accessory glands, exfoliated epithelial cells and cellular debris. The volume of discharges in asymptomatic women is about 1 to 6 mL per day and it is increasing during pregnancy. It must be considered that vaginal fluid can be contaminated and diluted by residual urine, sweat, or semen.

A “sanitary pad”, “sanitary napkin”, “menstrual pad”, or “pad” is an absorbent item worn by women and girls to absorb menstrual discharges. A pad is a type of feminine hygiene product that is worn externally, made from a range of materials, and which is typically disposable in nature. It is understood that other type of feminine hygienic items such as panty liners, which are similar to sanitary pads in terms of shape, functioning and wearability, are included into the definition of a sanitary pad according to the present disclosure.

A “microfluidic device”, “microfluidic chip” or “microfluidic platform” is generally speaking any apparatus which is conceived to work with fluids at a micro/nanometer scale. Microfluidics is generally the science that deals with the flow of liquids inside channels of micrometer size. At least one dimension of the channel is of the order of a micrometer or tens of micrometers in order to consider it microfluidics. Microfluidics can be considered both as a science (study of the behaviour of fluids in micro-channels) and a technology (manufacturing of microfluidics devices for applications such as lab-on-a-chip). These technologies are based on the manipulation of liquid flow through microfabricated channels. Actuation of liquid flow is implemented either by external pressure sources, external mechanical pumps, integrated mechanical micropumps, hydrostatic pressures or by combinations of capillary forces and electrokinetic mechanisms.

The microfluidic technology has found many applications such as in medicine with the laboratories on a chip because they allow the integration of many medical tests on a single chip, in cell biology research because the micro-channels have a similar size as the cells and allow such manipulation of single cells and rapid change of drugs, in protein crystallization because microfluidic devices allow the generation on a single chip of a large number of crystallization conditions (temperature, pH, humidity) and also many other areas such as drug screening, sugar testers, chemical microreactor or micro fuel cells.

Microfluidic devices or chips may comprise one or more valves. Any type of valve can be used in the frame of the present invention, such as motor-, screw-, solenoid- or pneumatic-actuated valves, these latter being preferred for manufacturing reasons (easily embeddable into a microfluidic device and less invasive in the frame of a medical device). The materials typically used for the production of the microfluidic device, including the valves, are soft materials, elastomers such as poly(dimethyl siloxane) (PDMS) or even hard materials such as thermoplasts, thermosets or glass. Using a transparent or translucent material advantageously allows to visually check a fluid flow process, if needed. Suitable ways of manufacturing the device are known in the art and can include etching, lithography, 3D printing and hot embossing, to cite some.

The expressions “film” or “thin film” relate to the thin form factor of an element of the device of the invention such as an electrode or a substrate. Generally speaking, a “film” or “thin film” as used herein relates to a layer of a material having a thickness much smaller than the other dimensions, e.g. at least one fifth compared to the other dimensions. Typically, a film is a solid layer having an upper surface and a bottom surface, with any suitable shape, and a thickness generally in the order of nanometers to millimeters, depending on the needs and circumstances, e.g. the manufacturing steps used to produce it and/or the scale of the overall system. In some embodiments, films according to the invention have a thickness comprised between 0.1 μm and 5 mm, such as between 5 μm and 5 mm, between 5 μm and 1 mm, between 10 μm and 1 mm, between 5 μm and 500 μm, between 50 μm and 500 μm between, between 50 μm and 150 μm, 100 μm and 500 μm or between 200 μm and 500 μm.

The expression “conductive track” refers to any film, path, stripe, strand, wire or the like which is electrically conductive in nature. For the sake of clarity, the word “electrode” is herein used to mean the distal part of a conductive track which is in direct contact with a sample under analysis. Conductive tracks according to the present disclosure are used to connect and/or close an electrical circuit, and are thus usually electrical connectors or “interconnects”. A conductive track is generally a metallic element that conducts an electric current toward or away from an electric circuit, but can be made of any suitable electrically conductive material, including but not limited to metals such as Au, Pt, Al, Cu and the like, as well as any alloy, oxides and/or combinations thereof; conductive polymeric materials; composite material such as polymeric materials embedding metal particles and/or metal strands or stripes, including insulating materials functionalized with electrically conductive flakes or fibers, for example carbon-filled polymers; liquid metals, including alloys or oxides thereof, such as gallium; electrically conductive inks; as well as any suitable combination thereof.

In the frame of the present description, a “biomarker” is a measurable indicator of some biological state or condition. Biomarkers are often measured and evaluated to examine normal biological processes, pathogenic processes, or pharmacologic responses to a therapeutic intervention. In the context of the present invention, aimed at assessing a pre-term birth (PTB) risk and/or premature rupture of membrane (PROM) risk, a “biomarker” preferably means a substance that indicates whether PTB and/or PROM risk is/are present. A “biomarker” includes chemical and/or biological species, the amount of which is present, increases and/or exceeds a certain threshold in subjects who are prone to PTB and/or PROM risk compared to normal healthy subjects. As used herein, the term “subject” refers to mammals, particularly humans, and even more particularly to pregnant women.

A non-exhaustive list of target biomarkers according to the invention includes small molecules, proteins, enzymes, antibodies, vitamins and the like. Exemplary target biomarkers include, but are not limited to, a growth factor, an oligopeptide, a polypeptide, an enzyme, an antibody or a fragment thereof, an antigen, any type of nucleic acid such as e.g. DNA, RNA, miRNA and the like, a hormone, a cytokine, a transmembrane receptor, a protein receptor, a serum protein, an adhesion molecule, a lipid molecule, a neurotransmitter, a morphogenetic protein, a differentiation factor, organic molecules, polysaccharides, a matrix protein, a cell as well as any combinations thereof.

Preferred biomarkers according to the invention may be selected from a list comprising Foetal Fibronectin (fFn), Insulin-like growth factor-binding protein 1 (IGFBP-1), Placental alpha microglobulin-1 (PAMG-1), Inflammatory cytochines including IL1A and IL1B, IL-2, IL-6 and IL-8, Tumor necrosis factor-alpha (TNF-alpha), C-reactive protein (CRP), Alpha-Fetoprotein (AFP) and Corticotropin-releasing hormone (CRH). Further biomarkers according to the inventive concept of the invention may be selected from a list comprising cystatin A (CSTA), monocyte/neutrophil elastase inhibitor (SERPINB1), squamous cell carcinoma antigen 1 (SERPINB3), squamous cell carcinoma antigen 2 (SERPINB4), interleukin-1 receptor antagonist (IL1RN), thioredoxin-1 (TXN), Zn-superoxide dismutase (SOD1), peroxiredoxin-2 (PRDX2), and glutathione S-transferase pi (GSTP1), epidermal fatty acid binding protein 5 (FABP5), annexin A3 (ANXA3), albumin (ALB), cysteine protease (CSTA), matrix metalloproteinases (MMPV, TIMP2 & TIMP1), Vitamin D binding protein (GC, group-specific component), α-fetoprotein, major basic protein, placental isoferritin, corticotropin-releasing hormone,

adrenocorticotropin, prolactin, human chorionic gonadotropin, C-terminal propeptide of procollagen, sialidase and municase. Additionally, cells or antigens derived thereof might be included in the list of suitable biomarkers according to the invention; examples of those additional biomarkers include herpes virus, vaginal gonococcus, chlamydia, group beta streptococcus, polymorphonuclear leukocytes and clue cells.

An “immunoassay” is a bioanalytic technique which exploits antibodies for the recognition of a target analyte. Antibodies are particularly suitable for this purpose, since (i) their bond with the target is sensitive and specific, (ii) they can be employed for identifying a large number of biomolecules, viruses and cells, (iii) the obtained bond is very strong.

The simplest immunoassay for proteins detection is the immunometric or sandwich assay. This technique involves capture antibodies (cAbs) that are immobilized to the substrate, and detection antibodies (dAbs), usually kept in solution with the analyte. The dAbs are conjugated to a label, e.g. a dye or a fluorophore. If the targeted analyte is present in solution, it will be sandwiched between the two antibodies and a signal (e.g. colorimetric or fluorescence) will be retrieved from the area where the cAbs are fixed.

A second method is commonly employed to perform assays of biomolecules: the sandwich enzyme-linked immunosorbent assay (ELISA). The recognition procedure again involves two antibodies with the same procedure as in the immunometric assay. The main difference is that enzyme-driven reactions are employed instead of labels. The enzymes are linked to the dAbs and their substrate is present in solution: if the cAb-analyte-dAb complex is formed, the enzyme catalyzes the substrate reaction that is producing a measurable signal (usually electrical or optical).

As used herein, a “readout” is an information output, typically a measurement output, displayed or otherwise presented to a user in a readable or analyzable/intelligible form. An information readout can be presented to a user in any suitable form, such as graphically, optically, acoustically etc. Once obtained, the information can be additionally or previously elaborated via a software or electronic devices before been presented to a user, such as through microprocessors or coupled computing devices such as computers, smartphones, tablets and the like.

The present disclosure describes a new disposable feminine hygiene product in the form of a sanitary pad or similar structure, which embeds a biosensing system as well as a microprocessor, battery and transceiver, hereinafter also referred to as “vaginal fluid monitoring device”. By analyzing vaginal discharges in the biosensor, and converting the results into a digital signal through an electrochemical detection and/or an optical signal through an optical detector, the device can monitor, sense and/or detect the presence into said vaginal discharges of one or more biomarkers, particularly biomarkers correlated with an increased risk of a PTB and/or PROM risks, as well as their concentration compared to a threshold indicative of an increased PTB and/or PROM risks.

A non-limiting advantage of the integrated device according to one or more embodiments is that the monitoring, analysis and reporting can be accomplished simply and with minimal steps and intervention by the user, which is generally a pregnant woman with or without a previous history of PTB and/or PROM risks. Once the sanitary pad is positioned and the integral diagnostic system has been activated, the process does not require further processing by the user. The possibility to obtain health information in a clean, non-invasive and user-friendly manner is attractive for many pregnant women. Furthermore it can provide frequent monitoring without the need of spontaneous attendance to clinical evaluation and extremely helpful for asymptomatic women or for those who don't recognize the risk indicators (e.g. the device could recognize the leakage of amniotic fluid due to infection, whose tears can be misinterpreted by the person as vaginal discharge or urine). Moreover it could be used in developing countries where no other means are currently available for PTB diagnostics, and can contribute to medical research on pregnancy care, furnishing a large amount of precious data on vaginal secretions or, for example, pointing out new correlations between different biomarkers concurrently tested.

The integrated feminine sanitary device provides for vaginal fluid collection using appropriately modified feminine hygienic pads and integrated microfluidics and microelectronics to provide the benefits of inexpensive, non-invasive, convenient and reliable health monitoring and diagnostics.

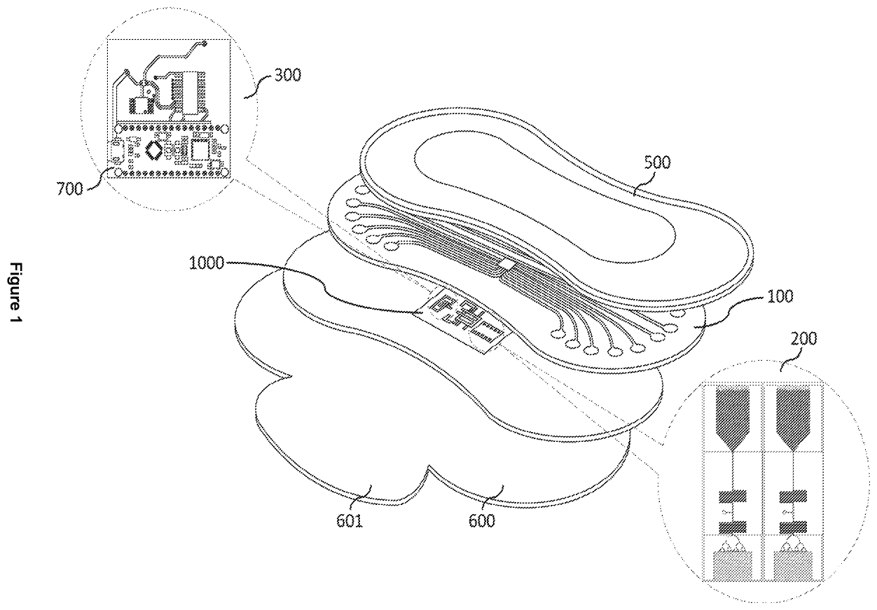

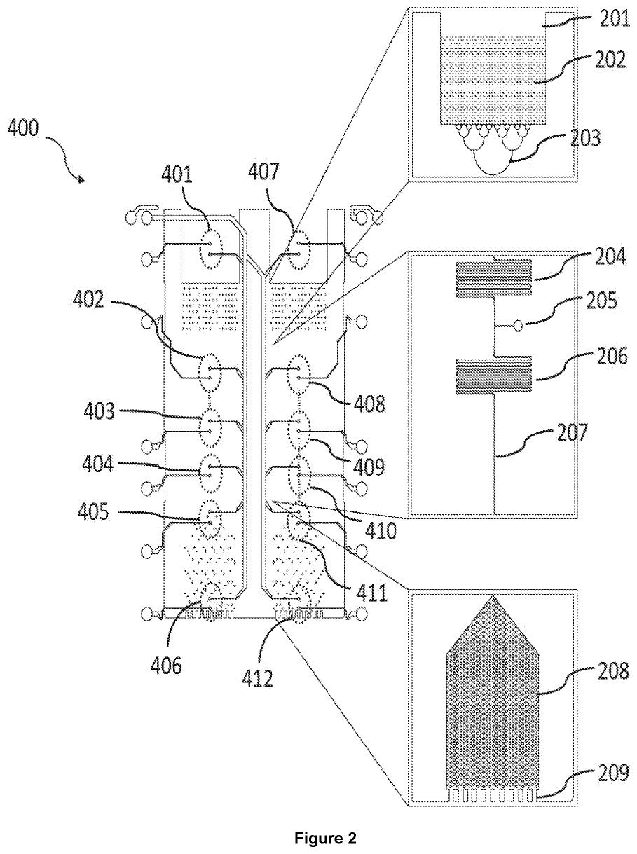

With reference to FIGS. 1 and 2, one embodiment of the device of the invention is shown as a vaginal fluid monitoring device embedded into a feminine sanitary pad, said device comprising a stack of the following elements:
a) An absorbent layer 100 configured to be in proximity to, and collect, a vaginal fluid; and
b) A biosensing system 1000 in fluidic connection with said absorbent layer 100, said biosensing system comprising:
i) A microfluidic chip 200 configured to perform an immunoassay to detect the presence and/or the concentration of at least one target biomarker comprised in a vaginal fluid;
ii) Means 300 for providing a readout of the presence and/or the concentration of said at least one target biomarker; and
iii) An electrode array 400 located along the microfluidic chip 200 configured to detect and analyze the flow of said vaginal fluid by impedance means
   wherein said immunoassay is configured to detect the presence and/or the concentration of at least one target biomarker indicative of a pre-term birth (PTB) risk and/or premature rupture of membrane (PROM) risk.

As depicted in the Figures, the device of the invention comprises preferably a top and bottom coverage sheets 500 and 600 for enclosing the functional elements in the sanitary pad, shaped and configured to resemble a classical pad for vaginal discharge absorption. The top coverage sheet 500 consists of a hydrophobic porous layer substantially made of biocompatible materials, providing a barrier with the skin, keeping it dry and allowing a vaginal fluid to be absorbed by the layer placed underneath, whereas the bottom sheet 600 is a breathable backsheet with preferably adhesive tape(s) 601 to fix the sanitary pad to underwear of a user. Underneath the top coverage sheet 500, an absorbent layer 100 is located, said layer being configured to be in proximity to a vaginal fluid and in any case in fluidic connection thereto, and to collect and direct the vaginal discharges to the below biosensing system 1000 once the sanitary pad is worn. To this aim, the absorbent layer 100 is tailored for maximizing the collection of a sample from the whole pad surface area, and is composed of absorbent materials such as cotton or preferably cellulosic materials such as paper.

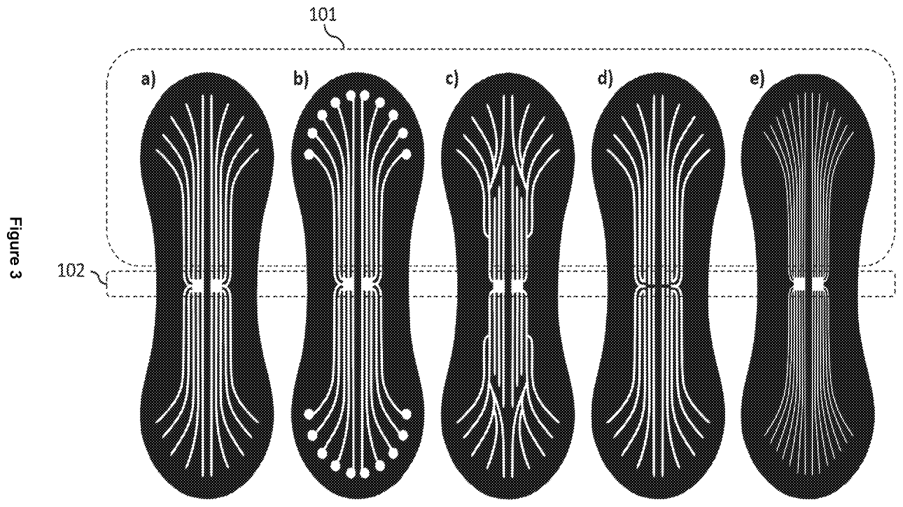

With reference to FIG. 3, in a preferred implementation of the invention, the absorbent layer 100 comprises a paper substrate patterned with fluidic paths 101. Paper-based analytical devices, μPADs, are currently gaining popularity in the bioanalytics field. These devices basically rely on the intrinsic capillary action of paper fibers and their natural tendency to absorb liquids. The flow is advantageously controlled by the presence of hydrophobic barriers inside the paper which are used to drag the fluids to desired areas of the system, particularly to a collection area 102, possibly comprising inlet openings, fluidically coupled with the biosensing system 1000 placed underneath. This configuration is clearly advantageous for the development of point-of-care diagnostic systems, paper being a cheap and disposable material, which can be easily processed and is available in large quantities. Besides, its natural capillary effect enables the design of passive (micro)fluidic devices with much easier fabrication processes.

For the fabrication of μPADs, the patterning of the channels 101 can be mechanical or chemical based. Mechanically obtained fluidics is simply based on channel confinement by paper cutting. The desired shape can be patterned by a computer-controlled knife plotter or laser cutting. The second option involves the chemical modification of the paper in order to insert hydrophobic barriers. The paper area confined by the barriers constitutes the (micro)fluidic path. Deposition techniques can be direct or indirect.

Direct treatments rely on the placement of the hydrophobic agent only in the desired region, not affecting the paper fluidics area. Available methods are wax printing, inkjet printing, writing, flexography printing, plotting. Commonly employed printable inks are wax, alkyl ketene dimer (AKD), silicone, polystirene, hydrophobic sol-gels, UV-curable inks. Advantageously, a modification of the paper in order to insert hydrophobic barriers may provide a protective barrier impermeable to fluids (could be a plastic or other hydrophobic materials) and may additionally serve to protect the biosensing platform 1000 as well as any embedded microelectronics and/or detectors 300.

As said, the collection area 102 leads the collected vaginal discharges towards a first element of the biosensing system 1000, namely a microfluidic chip 200 expressly designed to perform an immunoassay to detect the presence and/or the concentration of at least one target biomarker comprised in a vaginal fluid. A microfluidic chip 200 according to the invention can be manufactured through methods known in the art, such as (photo)lithography, molding, etching, laser cutting and the like of a solid support substantially composed of one or more materials such as glass, polymeric materials such as plastics (e.g., polystyrene, polypropylene, polycarbonates, polysulfones, polyesters, cyclic olefins and so forth) as well as soft polymers like polydimethylsiloxane (PDMS), acrylic elastomers, rubber, polyurethane (PU), polyvinylidene fluoride (PVDF) or similar. Depending on the needs and circumstances, the microfluidic chip 200 can be further encapsulated with one or more materials as e.g. those listed above to guarantee physical isolation from the rest of the fluid monitoring device, as well as for instance sterility of antibodies used for the immunoassay steps.

The microfluidic chip 200 can comprise one or more microfluidic channels, either straight or branched ones, and may include turns or serpentines, as well as one or more reservoirs for e.g. hosting reagents or antibodies (see for instance FIG. 2). In particular, in order to perform an immunoassay to detect the presence and/or the concentration of target biomarkers indicative of an increased risk of PTB and/or PROM, in embodiments of the invention the microfluidic chip 200 comprises at least one portion, such as a channel and/or a reservoir, comprising at least one antibody binding to one or more target biomarker indicative of PTB and/or PROM risks, such as at least one antibody binding to one or more biomarkers selected from a group comprising Fetal Fibronectin (fFn), Insulin-like growth factor-binding protein 1 (IGFBP-1), Placental alpha microglobulin-1 (PAMG-1), Inflammatory cytochines including IL-1, IL-2, IL-6 and IL-8, Tumor necrosis factor-alpha (TNF-alpha), C-reactive protein (CRP), Alpha-Fetoprotein (AFP) and Corticotropin-releasing hormone (CRH). Further suitable antibodies can be selected from antibodies binding to one or more biomarkers selected from a group comprising cystatin A (CSTA), monocyte/neutrophil elastase inhibitor (SERPINB1), squamous cell carcinoma antigen 1 (SERPINB3), squamous cell carcinoma antigen 2 (SERPINB4), interleukin-1 receptor antagonist (IL1RN), thioredoxin-1 (TXN), Zn-superoxide dismutase (SOD1), peroxiredoxin-2 (PRDX2), and glutathione S-transferase pi (GSTP1), epidermal fatty acid binding protein 5 (FABP5), annexin A3 (ANXA3), albumin (ALB), cysteine protease (CSTA), matrix metalloproteinases (MMPV, TIMP2 & TIMP1), Vitamin D binding protein (GC, group-specific component), α-fetoprotein, major basic protein, placental isoferritin, corticotropin-releasing hormone, adrenocorticotropin, prolactin, human chorionic gonadotropin, C-terminal propeptide of procollagen, sialidase and municase. Additionally, further suitable antibodies can be selected from antibodies binding to one or more biomarkers selected from a group comprising antigens derived from herpes virus, vaginal gonococcus, chlamydia, group beta streptococcus, polymorphonuclear leukocytes and clue cells.

In a preferred embodiment, the microfluidic chip 200 is configured to operate in a passive capillary regime, that is, the design of the device is optimized to allow a vaginal fluid flow along the microfluidic channels and/or reservoirs without the need of any externally imparted pressure force, but only exploiting the capillary interactions between the fluid and the microchannels' internal walls. Exploiting capillary forces, this configuration avoids the need of externally imparted pressure forces such as (micro)pumps or valves, thereby hugely facilitating manufacturing and implementation of the entire system in a cost-effective manner inside a sanitary pad. One exemplary, non-limiting design of a microfluidic chip operating in a passive capillary regime is provided in the Example section and FIG. 2, and will be described later in more details.

In embodiments of the invention, the microfluidic chip 200 is based on a flexible platform. The flexibility accommodates the normal movements of the user when the feminine hygiene integrated device is worn. In one or more embodiments, the microfluidic chip 200 is a paper-based system. As described above with reference to the absorbent layer 100, paper-based microfluidic devices are a promising and cheap technology in developing analytical devices for point-of-care diagnosis. The paper-based microfluidics provides a novel system for fluid handling and fluid analysis, for a variety of applications including health diagnostics and monitoring, as well for other fields. Some of the reasons paper is an attractive substrate for making microfluidic chips include: it is ubiquitous and not expensive, it is compatible with many chemical/biochemical/medical applications; and takes advantages of capillary forces for fluid flow, without the need of applying external forces such as pumps. Paper-based microfluidic chips are low-cost, easy-to-use and disposable.

The microelectronics 700 can also be mounted on a paper platform (or other flexible platform such as flexible PCBs), positioned for interaction and communication with the biosensing system 1000. The electrical parts are becoming cheaper, lower powered, smaller in feature size and more advanced in their capabilities due to a shrinking of electronics, often down to a nano-scale resolution in features sizes, thus permitting their integration into thinner and smaller items.

The biosensing system 1000 comprises means 300 for providing a readout of the presence and/or the concentration of at least one target biomarker, which are implemented as a detection system for identification and/or characterization of said biomarker(s) in a vaginal fluid, based on the results of the immunoassay performed in the microfluidic chip 200.

In one embodiment of the invention, a readout can be an electrochemical readout, and the detection system is an electrochemical detection system. Electrochemical analysis is ubiquitous in analytical laboratories but it usually utilizes complicated and expensive instruments, which requires special trained technicians. However, for use in e.g. the field of home-care, there remains a need for analytical devices that are inexpensive, disposable, portable and easy to use. Electrochemical paper based analytical devices as well as screen printed electrodes, have recently been explored as the basis for low-cost, portable devices, especially for use at the point of care. They generally employ a printed Ag/AgCl pseudo-reference electrode, but other types materials has been used to produce the electrode, often coating it for a specific reaction. Typically, the coating comprises an “anchor” nucleic acids (DNA, RNA) and/or proteins (including antibodies) in order to allow for a quantitative, reagent-less, electrochemical detection of target analytes such as biomarkers, preferably directly in unprocessed clinical samples (such as vaginal discharges). Such paper based or flexible platforms can be used to screen for a large number of disease states and conditions.

This platform can also be used for an antigen/antibody or antibody/antibody detection, which is the preferred setting in the frame of the present disclosure. In this case, the anchor is an antibody specific for a target partner, such as a biomarker or a biomarker/antibody conjugate, which has been assembled on to a working electrode. Binding of the target partner to the biosensor changes the amount of current to the electrode, thereby altering e.g., the impedance of an electrical circuit. This method can be used via the presence of an electrochemical conjugated label on the dAB or not. The coated working electrode may be connected to a potentiostat together with a counter electrode and a reference electrode. When fabricating these electrochemical biosensors, a background square wave voltammetry is performed, this background measurement being converted and read by the potentiostat, and registered and saved in a microcontroller/microprocessor. This enables the microprocessor to register the change the current relative to this background measurement when the target molecule binds to its partner antibody. When a vaginal discharge reaches the biosensor, and if the target biomarker and/or biomarker/antibody conjugate is present, the peak at the same voltage which is measured by the potentiostat and registered by the microprocessor will decrease. The magnitude of this change is related to the biomarker and/or biomarker/antibody conjugate concentration. However, an electrochemical readout according to the invention is not limited to the above described embodiment, and to the contrary it generally exploits changes in any electrical properties measurable through any electroanalytical method, such as potentiometric, amperometric or conductimetric detection approaches, said changes being related to the presence and/or the concentration of a target analyte, particularly (a) target biomarker(s), even more particularly (a) target biomarker(s) indicative of a risk of PTB and/or PROM.

In additional or alternative embodiments of the invention, a readout provided by the biosensing system can be an optical readout, and the detection system is an optical detection system. Optical biosensors offer great advantages over conventional analytical techniques because they enable the direct, real-time and possibly label-free detection of many biological and chemical substances. Their advantages include high specificity, sensitivity, small size and cost-effectiveness. Optical detection is performed by exploiting the interaction of the optical field with a biorecognition element. Optical biosensing can be broadly divided into two general modes: label-free and label-based. Briefly, in a label-free mode, the detected signal is generated directly by the interaction of the analyzed material with a transducer. In contrast, label-based sensing involves the use of a label and the optical readout is then generated by a colorimetric, fluorescent or luminescent method, such as an enzyme-based colorimetric, fluorescent or luminescent chemical reaction.

In one embodiment according to the invention, means for providing an optical readout comprises an optical detector for detecting a signal generated by an immunoassay. An optical detector according to the invention can be configured as a compact analytical device containing an illumination element operably coupled with a transducer. In particular, in one embodiment the optical detector comprises a light source, such as a laser or LED source, operably coupled with one or more photodetectors such as photodiodes. The basic objective of an optical detector as described herein is to produce a signal which is proportionate to the concentration of a measured substance (in this case, a target biomarker).

In embodiments of the invention, the biosensing system 1000 can test for a variety of different biomarkers. It may be desirable to analyze one or more reference biomarkers to determine the amount of a reference biomarker and compare this to a target biomarker, where the concentration of the referenced biomarker is generally known. This permits to use the ratio of the referenced biomarker to the target biomarker to determine the concentration of said target biomarker without knowing the volume of the vaginal fluid being tested. Typical reference biomarkers include known methods such as those used to determine electrolyte balance. Some biomarkers found in vaginal fluid may degrade quickly due to enzymatic or other forms of decomposition or breakdown, and storage, preservation, chemical reacting, or other chemicals, materials or components, may be included in the biosensor 1000 to preserve the desired information provided by the biomarkers.

In a system according to the invention for collecting and electrochemically analyzing vaginally discharged fluids, once the vaginal fluid is collected through the first absorbent layer 100 and directed through a channeling system 101-102 towards one or multiple sites of a biosensing system 1000, the detection of one or more target biomarkers takes place thanks to the specific interaction between said biomarker(s) and partner molecules coated on sensing electrochemical electrodes. The biosensing system 1000 is coupled to three sensing electrodes: a working electrode, a counter electrode and a reference electrode. The reference electrode has a known reduction potential and its only role is to act as reference in measuring and controlling the working electrode's potential and at no point does it pass any current. These three electrodes make up the classical three-electrode electrochemical system. Binding one or more target biomarkers from the vaginal fluid causes a change in the impedance or current in the working electrode. This will either increase or decrease the current detected by a coupled microprocessor 700. The relative change in current relates to the amount of a target biomarker detected at the detection site. This information/data can be stored in the microprocessor 700, and possibly encrypted and eventually sent to a receiving device such as a smart phone through e.g. wireless means (e.g. Bluetooth modules). The data received by the microprocessor 700 is used by embedded software to compare changes in e.g. electric impedance to a reference condition. The microprocessor 700 may be programmed to sleep with a specific time interval and to it wake up to run a test. If the impedance detected in the system has not changed, it will sleep again and wake up after another time interval. If it detects a change and therefore the targeted biomarker, it will e.g. encrypt both raw data and processed data and send the information to the device once the device is near.

In an alternative embodiment, a colorimetric detection system is used, which in case of biomarker detection will lead to a color change shown in e.g. color changing pads. For detecting quantities of certain target biomarker levels, where only the presence is not enough information, a colorimetric reference system will be needed (e.g. provided in a kit comprising a package) similar to what is used for reading amounts of H+when doing pH measurements.

To ensure constant and equal flow rate through the microfluidic channels of the microfluidic chip 200, therefore ensuring an equal amount of vaginal fluid delivered to the biosensing system 1000 (electrochemical/colorimetric etc.), said absorbent layer 100 is configured to keep a constant flow of a vaginal fluid inside said microfluidic chip 200. A detailed description of some implemented embodiments of said configurations are provided in the Example section. With the same aim and purpose, the microfluidic structures of the microfluidic chip 200 are also configured to keep a constant flow of a vaginal fluid along the chip up to the detection system (electrochemical/colorimetric etc.) 300. Again, a detailed description of some implemented embodiments of said configurations are provided in the Example section. Further, the microfluidic chip 200 may include a microcollector, preferably equipped with a membrane filter, which feeds the vaginal discharge into the microfluidic inlet channels. The vaginal discharge membrane filter can filter e.g. plasma, cells and/or a protein, acting as a “purification” system.

The biosensing system 1000 comprises in addition an electrode array 400 located along the microfluidic chip 200 and configured to detect and analyze the flow of said vaginal fluid by impedance means (see for instance FIG. 2). In embodiments, a plurality of “sensing sites” (401-412 in the non-limiting embodiment depicted in FIG. 2) are distributed along the microfluidic path, each site consisting of a pair of electrodes, such as golden planar electrodes. By measuring the impedance between the two electrodes of each site 401-412 it is possible to determine the presence of a liquid at that particular position inside the chip 200. Real-time monitoring of the chip filling is necessary for carrying out the bioanalysis. Moreover, being the device of the invention a wearable system, it must periodically check whether a sample has been collected or not. Once the presence of the analyte is assessed, the analysis can be conducted. Furthermore, in the frame of integrated analysis and wireless data sharing, the electrode array 400 results convenient in terms of energy savings to keep quiescent the unnecessary electronics. The positive response of the real-time flow monitoring would activate the other modules, starting the analytical process.

Measurements on sample conductivity can also be useful for investigating the quality of the collected vaginal secretions in terms of dilution, e.g. by sweat or urine. In fact, physical characteristics of the collected vaginal secretions such as the viscosity can be estimated based on the obtained flow and on other measurable parameters. The flow is constant due to the engineered absorbent (paper) layer 100 and the design of the microfluidic chip 200. Its value is measured by the electrode array 400 and it depends on the viscosity of the liquid. The thicker the liquid, the slowest inside of the system and the less diluted a target biomarker is expected to be in the system. The temperature and pH play a similar role in the system. The higher the temperature, the more a biomarker will be expected to be diluted in vaginal secretions with sweat. In such a way, through the processing and the analysis of the retrieved flow measurement, it shall be possible to calculate the dilution of the target biomarkers in vaginal fluids, thereby obtaining a calibration step functional to determine the concentration of target biomarkers.

As variations in pH and/or temperature could impact the target proteins of interest, and variations of pH are also directly related to infections that could case PTB and/or PROM, (an) additional pH sensor and/or temperature sensor is (are) contemplated within the system in some embodiments.

Electrode components of the electrode array 400 preferably comprises a distal, end electrode portion, such as a pad, configured to directly interface the microfluidic chip 200. Conductive tracks and/or electrode pads of the electrode array can be made of any suitable electrical conductive material, including but not limited to metals such as Au, Pt, Al, Cu, Pt—Ir, Ir, and the like, as well as any alloy thereof, oxide thereof and combinations thereof, composite metal-polymer materials, such as Pt-PDMS composites or Pt—Ir-PDMS composites or Ir-PDMS composites and so forth, as well as conductive polymers such as poly(3,4-ethylenedioxythiophene) polystyrene sulfonate (PEDOT:PSS) or polypyrrole (PPy). In one embodiment, the electrodes are made of non-toxic and biocompatible materials. Electrode and/or electronic component or portions thereof (e.g. conductive tracks or electrode pads) can be placed on or within a support with any suitable means such as for instance photolithography, electron beam evaporation, thermal evaporation, sputter deposition, chemical vapour deposition (CVD), electro-plating, molecular beam epitaxy (MBE), inkjet printing, stencil printing, contact printing, transfer printing or any other conventional means known in the art. In embodiments, conductive tracks are encapsulated later on to avoid short circuits and failure thereof, i.e. passivated whilst leaving the electrode pads exposed through connecting vias.

In a set of embodiments according to the invention, the electrodes and/or conductive tracks comprised in the electrode array 400 are compliant electrodes and/or conductive tracks. A “compliant electrode” is any structure or element able to deliver an electric current, and adapted to change its shape according to the shape change of the support it adheres to, without substantially compromising mechanical and/or electrical performances. The term “compliant” is intended to include any conformable structure which is compressible, reversibly compressible, elastic, flexible, stretchable or any combination thereof. Examples of compliant electrodes known in the art include metal thin-films (including patterned electrodes, out-of-plane buckled electrodes, and corrugated membranes), metal-polymer nano-composites, carbon powder, carbon grease, conductive rubbers or conductive paints, a review of which is provided in Rosset and Shea (Applied Physics A, February 2013, Volume 110, Issue 2, 281-307), incorporated herein in its entirety by reference. As it will be apparent to those skilled in the art, built-in multilayers or stacks of several layers of any of the above polymeric, composite, metallic and/or oxide materials, as well as combinations thereof, are encompassed in the definition of compliant interconnect.

As anticipated, the biosensing system 1000 preferably further comprises a microprocessor 700 configured for processing a signal generated by said biosensing system 1000, such an electrical impedance signal and/or an optical signal.

As it will be apparent from the previous discussion, one additional aspect of the invention relates to a method for detecting at least one target biomarker comprised in a vaginal fluid, said method comprising the steps of:
allowing a vaginal fluid monitoring device according to the invention to collect a vaginal fluid; and
obtaining a readout, such as an optical and/or an electrochemical readout, of the presence and/or the concentration of said at least one target biomarker, thereby determining the presence and/or the concentration of said at least one target biomarker in a vaginal fluid. The presence of said at least one target biomarker is, according to the core of the invention, indicative of a pre-term birth (PTB) risk and/or premature rupture of membrane (PROM) risk, and is preferably but not exclusively selected from a group comprising fetal Fibronectin (fFn), Insulin-like growth factor-binding protein 1 (IGFBP-1), Placental alpha microglobulin-1 (PAMG-1), Inflammatory cytochines including IL-1A, IL1B, IL-2, IL-6 and IL-8, Tumor necrosis factor-alpha (TNF-alpha), C-reactive protein (CRP), Alpha-Fetoprotein (AFP) and Corticotropin-releasing hormone (CRH), cystatin A (CSTA), monocyte/neutrophil elastase inhibitor (SERPINB1), squamous cell carcinoma antigen 1 (SERPINB3), squamous cell carcinoma antigen 2 (SERPINB4), interleukin-1 receptor antagonist (IL1RN), thioredoxin-1 (TXN), Zn-superoxide dismutase (SOD1), peroxiredoxin-2 (PRDX2), and glutathione S-transferase pi (GSTP1), epidermal fatty acid binding protein 5 (FABP5), annexin A3 (ANXA3), albumin (ALB), cysteine protease (CSTA), matrix metalloproteinases (MMPV, TIMP2 & TIMP1), Vitamin D binding protein (GC, group-specific component), α-fetoprotein, major basic protein, placental isoferritin, corticotropin-releasing hormone, adrenocorticotropin, prolactin, human chorionic gonadotropin, C-terminal propeptide of procollagen, sialidase, municase, as well as antigens derived from herpes virus, vaginal gonococcus, chlamydia, group beta streptococcus, polymorphonuclear leukocytes and clue cells.

### Examples

In the following, some exemplary, non limiting examples of some implemented embodiments of the device of the invention are provided.

The present invention proposes a new device for PTB risk assessment which enables non-invasive, frequent, possibly at-home monitoring though a wearable device. The system is a “smart” pad, shaped as a common sanitary pad with embedded sensing and electronics for wireless data sharing. It collects vaginal secretions and measures the concentration of one or multiple biomarkers. In particular, the device contains two functional layers: one for vaginal fluid collection and displacement to the sensing area; a second one with the sensing system and the read-out components. This device opens the possibility of PTB monitoring with a comfortable solution, without clinical evaluation, avoiding invasive tests and screening also asymptomatic women.

A miniaturized fluidic device perfectly matches the requirement of portable bio-analytics with small sample amount. A microfluidic chip has been designed and a dedicated microfabrication process developed and optimized, along with the choice of materials for guaranteeing its functional operation. Sample pre-filtering, advantageous for removing large particulates in vaginal fluid which would compromise the measurements, can be embedded on chip.

A second module has been conceived, in order to gather the sample from the whole pad area and to minimize the losses occurring while the liquid is passively displaced to the inlets of the microfluidic chip. An useful approach for this application is to directly pattern channels into the paper layer that is collecting the biological sample. The fabrication processes of paper microfluidic has been therefore investigated, and a specific procedure chosen and tailored to the compelling needs. Furthermore, the paper fluidic network has been designed to engineer the collection and effectively pair this element with the microfluidic chip.

The last module added to the device prototype is a miniaturized system for real-time monitoring of the liquid filling level. While the device is worn by the user, this system is continuously checking for the sample collection and decides when it is necessary to initiate the bio-analytic sensing procedure. An electronic read-out is interfaced to planar electrodes which are microfabricated onto the chip along the fluidics pattern. Eventually, the portable electronic board design was adapted in order to integrate the three developed modules together. The microfluidics filling level can indeed be monitored in real-time, with the chip embedded with the paper collection pad; the data may be wirelessly communicated to a smartphone application.

Capillary Microfluidic Chip

The miniaturization of the fluidic pathways provides the advantage of large surface-to-volume ratios: surface effects, as capillarity, are dominant at the microscale. Interestingly, microfluidic chips can be designed to passively collect liquid sample just through capillary action.

Immunoassays are widely employed in medical diagnostics, for example the commercial kits for preterm birth testing (fFN, phIGFBP and PAMG-1) are based on immunoassays. The most popular immunoassay at-home test is the lateral flow assay on human chorionic gonadotropin, e.g. the pregnancy test. Several studies are aiming at integrating immunoassays on microfluidic chips. Miniaturization of the analysis is particularly convenient since it reduces the consumption of samples and reagents. In addition, it is intrinsically more sensitive and fast due to the low mass-transport times at small scale. It can enable the integration of multiple functions on the same portable device, allowing to perform at home the same analysis which is carried out in a laboratory, avoiding a large number of steps or the need of specialized operators. Point-of-care diagnostics would be especially useful in cases where continuous monitoring is required and fast responses are needed, e.g. cardiac diseases and preterm birth diagnosis.

In order to make the on-chip analysis procedure user-friendly, the most suitable option for the design of the microfluidics was capillary fluidics. Capillary fluidics is useful for the automation of sandwich immunoassays, making them user-free. This approach enables the development of the so-called one-step immunoassays, i.e. to build a sensing platform where the only operation to be carried out by the user is the addition of sample to the main inlet. Successively, the liquid displacements, all the required additions of reagents and the responses generating the results are automated and predetermined by the chip design itself. For this purpose, the co-reagents are to be immobilized in the chip in dry form.

ELISA immunoassay are often performed successively adding the required reagents, leading to long and complex procedures. Capillary chips are designed to avoid the need of specialized user to carry out the analysis. It is possible in fact to build capillary circuits with multiple elements (pumps, resistors, valves) that can perform complex operations, with limited operator intervention, low footprint on chip and fast responses. This design technique enables to have precise tuning of the flow rate and volume capacity without active pumps or valves, since these parameters are only defined by the fluidic circuit geometry, size and materials. Microfluidic circuits can even be fabricated as open or suspended channels. Furthermore, capillary chips can be easily fabricated on transparent substrates that are compatible with optical readouts. However, since the flow rate is intrinsically defined by the design of the chip, it cannot be tailored externally or varied in time; moreover process variations can impact dramatically the device operation.

The concept of one-step immunoassay perfectly matches the requirements of the device of the invention: the chip can be embedded in a sanitary pad and as soon as the fluid sample wets the loading pad it is passively pumped inside the fluidic circuit. In this way, while the sample is serially owing through the different functional elements on chip, the analytical steps are successively performed and the results are automatically obtained without any user action.

Microfluidic Capillary Chip Layout

The layout of the microfluidic capillary chip used in an implemented embodiment of the invention is depicted in FIG. 2.

For the fabrication of the microfluidic channels, SU-8 photolithography on glass substrate was employed. Furthermore, the sealing procedure is carried out with PSA adhesive. Moreover, electrodes are placed along the fluidic circuit in order to perform real-time flow monitoring through impedance measurements.

The implemented, non-limiting embodiment of the microfluidics layout is displayed in FIG. 2. The inlet of the chip is made up of a square loading pad 201 (4.5 mm×4.5 mm) where a paper filtering membrane is to be placed. The membrane is required in order to filter the vaginal discharge sample, removing cell debris and other large size particles that can clog the chip microfluidics and interfere with the immunoassay. Since filtering membranes for vaginal secretions are not available on the market, blood filters, commonly employed to obtain plasma form whole blood, are employed for developing the device prototype.

After loading, the sample is dragged into the chip by the sample collector 202, a reservoir (3 mm×4.5 mm) with multiple vertical microstructures in regular series along asymmetric lines, able to collect the sample by capillarity. The structures shape is rectangular with rounded edges, with dimensions of 20 μm×80 μm. This design is proven to be effective in minimizing the bubble formation.

Delay valves 203, made of semicircular hierarchical structures, connect the collector 202 with the first flow resistor 204 guaranteeing homogeneous filling without bubbles formation. Moreover, their structure with branches of increasing dimensions (30-45-60 μm) assures that the sample is dragged inside the circuit only if its filling front is distributed along the whole collector width: the liquid, in fact, can only access the following layer if both the branches are filled. Successively, a secondary inlet 205 (a circular reservoir 400 μm in diameter) is laterally connected to the main circuit between the first and a second resistor 206. Its function is to collect the detection antibodies, deposited here in dry form. Resistors 204-206 are obtained designing serpentine channels (width 60 μm). They are useful to tailor the flow rate, filling time and total volume of the system. Furthermore, the second resistor 206 is essential to delay the liquid sample in order to enable its mixing with the detection antibodies collected from the secondary inlet 205.

A long straight channel 60 μm wide is the area were the immunoassay takes place (reaction chamber 207). Lines of capture antibodies and control lines are to be deposited onto the sealing in correspondence of this region. At the end of the microfluidic circuit a capillary pump 208 is exploited to pump the liquid inside by capillary force. It is made up of a 22 mm² area covered by cylindrical pillars 50 μm in diameter, disposed asymmetrically in parallel lines. Here the presence of numerous vertical pillars offers a very large superficial area that is able to exert a large capillary force. The smallest the free area for liquid filling, the highest the capillary pressure (Young-Laplace pressure). The strongest pumping action is though obtained with small and close features. Besides, the use of microstructures inside the capillary pump has the scope of minimizing air entrapment and bubble formation.

Eventually, vents 209 are placed at the outlet in order to expose the sample to the ambient air when the chip is sealed. If the chip was closed the air trapped inside would prevent the liquid from entering. Two parallel microfluidic circuits as the one described here above might be present in the same chip.

Planar gold electrodes are patterned at the fluidics bottom, directly on the glass substrate, in order to perform impedance measurements for liquid filling monitoring system. Pairs of circular electrodes 401-412 (100 μm in diameter) are placed at various points along the microfluidic circuit. In each pair one electrode is the measuring one, connected to an external pad, and the other is the reference. Reference electrodes are connected internally and routed to a common external pad. External pads are circles of 800 μm diameter: the routing wires are 30 μm or 200 μm in size. Six sensing sites are inserted along the fluidics path: in correspondence of loading pad 201, first resistor 204, second resistor 206, reaction chamber 207, pump inlet 202, pump outlet 208. Unconnected pads on the left are inserted in order to calibrate the impedance measurements: two connected pads (up-left in figure) are needed for closed-circuit calibration, two unconnected ones (bottom-left) for open-circuit calibration. Metal crosses delimit the area that is to be diced.

The manufacturing process basically consists in two layers patterning onto a float glass wafer; electrodes deposition through lift-off corresponds to the first lithographic mask. The second mask concerns the fabrication of the microfluidic channels in SU-8. Eventually the chips are individually sealed with PSA adhesive and the wafer is diced in order to obtain eight chips.

Capillary Microfluidic Flow Characterization

In order to test the capillary fluidics before irreversibly sealing the chips it is necessary to make the SU-8 walls hydrophilic enough in order to assure the generation of the Young-Laplace pressure. For this purpose the chip is subjected to Oxygen plasma treatment, known in the literature to be effective for enhancing the SU-8 wettability, with a contact angle lower than 5°. Taking advantage of the plasma activation, a first set of tests is performed before sealing the chips. All the different devices are properly filling, after a few tens of seconds the water reaches the reaction chamber and in about 5 minutes the pump is completely filled. The timings are of course influenced by a significant evaporation of the water sample. The plasma activation is effective for about 40 min, successively the channel walls recover their hydrophobicity and the liquid is not filling the chip. The chips are subsequently sealed with hydrophilic PSA adhesive. In this configuration the capillary pressure is assured by the presence of the hydrophilic surfaces of PSA and glass substrate. Glass has high water wettability, with a contact angle below 20°. The bond is proven to be effective since there is no liquid leakage. Sealed chips are tested even after 5 months and their sealing quality is not degraded.

After sealing with PSA, experiments on the chip filling are carried out delivering 10 μL of deionized water on the loading pad of the device. Test are performed with and without inserting paper membranes. In particular, two membranes are available, with different thickness and pore size (membrane 1-330 μm thick, 20 μm average pore size vs membrane 2-140 μm thick, 0.8 μm average pore size).

In both cases (with and without paper) the water completely fills the reaction chamber 207 in about 35-40 seconds, with a flow rate of 300-350 nL*min⁻¹. The pumps are filled up to the PSA edge in correspondence of the vents at the outlet: the liquid reaches this level after 2 min:30 s (370 nL*min⁻¹; flow rate up to reaction chamber: 340 nL*min⁻¹) in the chip without filter, after 4 min:15 s (220 nL*min⁻¹; flow rate up to reaction chamber: 350 nL*min⁻¹) with membrane 1 and after only 1 min:10 s (850 nL*min⁻¹; flow rate up to reaction chamber: 300 nL*min⁻¹) in the one with paper membrane 2. The chip volume up to the reaction chamber 207 is equal to 200 nL and the total volume capacity is 920 nL. It is noticeable that the flow rate for filling the chip up to the reaction chamber 207 is comparable in the three cases. It is the time required for dragging the liquid sample to the active area where the immunoassay is performed. In other words, about 40 seconds after the sample reaches the inlet the analysis is started. The total time required for filling the entire volume is instead remarkably influenced by the filtering membrane characteristics. Water starts evaporating from the pump about 10 min after the filling of the whole chip volume. The evaporation affects first the pump 201 and the sample collector 202. The fluidic channels (resistors 204-206 and reaction chamber 207) are dried successively, after 45-60 min. Thus the sample is retained in the reaction chamber 207 available for being analyzed for about one hour, more than enough for obtaining the test result. One-step immunoassays in fact typically require about 10 minutes.

Paper Collection System

In the present work, paper fluidics is needed in order to engineer the sample collection inside the sanitary pad and to effectively drag it to the sensing area. The selected fabrication process for this purpose is wax printing, since it is a fast, cheap and scalable fabrication procedure. Indirect techniques are not here preferable since they can modify the paper fluidic composition, contaminating the sample and though interfering with the diagnostic process. Moreover, suitable channel dimension for the present application are on the order of hundreds or thousands of micrometers, easily obtainable with direct deposition techniques, as common printing.

Wax Channels Patterning

Wax printing is one of the most employed methods for μPADs fabrication, being inexpensive, easy and high-throughput. Furthermore, processing devices developed for diagnostics it is desirable not to modify the paper constituting the hydrophilic channels, in order to avoid contaminations of the sample or of the immunoassays reagents. The procedure is simple and fast, involving basically only two steps. First, a wax printer is employed to print the desired pattern onto a paper sheet. The printer is working exactly as a common commercial printer, employing a wax-based ink instead of an aqueous one. The wax ink is transferred molten onto the paper and re-solidifies onto the surface. Subsequently, a reflow step at high temperature is needed to melt the wax and let it penetrate throughout the whole paper thickness. In this way, hydrophobic wax barriers are obtained inside the paper, delimiting hydrophilic channels. The desired layout of the channels array can be designed using common design programs.

Absorbent Collection Layer Layout

In order to perform real-time monitoring of PTB biomarkers levels, the user shall wear the smart pad device, shaped as a common feminine sanitary pad. The pad is collecting vaginal secretions from a large area, in order to maximize the chances of gathering sample while the person is wearing it. Nevertheless, it is of primarily importance to efficiently drag the collected sample to the biosensing system, i.e. to the loading pads of the microfluidic capillary chip. For this reason, the absorbent layer inside the sanitary pad consists in a fluidic network, whose channels are shaped in paper with the process described here above. Most of the exposed surface of the collection layer is hydrophobic due to wax treatment, thus only the channels area is capable of absorbing liquids. The channels, therefore, are to be designed in a way that minimizes the unwanted sample retaining by the paper and maximizes the displacement of vaginal secretions to the inlets of the sensing system. The best approach is to connect the center of the pad, which is to be in direct contact with the microfluidic chip inlets, with the peripheral areas through straight channels. The use of a tree structure for shaping the channels network, in fact, is not efficient for this application since branches deviate part of the sample volume from the path that is connecting it to the central reservoir. In other words, the best option is to provide to each drop of liquid deposited on the pad a unique direct path to the center.

The simplest approach is to design two central reservoirs and to use straight channels to connect them to the pad periphery (FIG. 3a). The reservoirs have the same dimensions of the loading pads on the capillary chip. The square paper filtering membranes are to be in direct contact with the loading pads on the chip on one side and with the collection pad reservoirs on the other one. The chip is embedded in the layer underneath. Considering that the paper capacity is approximately equal to 400 mL*m⁻², the hydrophilic area of this first design is capable of retaining 620 μL of sample. Further modifications of this radial network with parallel separated channels are introduced in other design versions. The pad shown in FIG. 3b shows circular distal reservoirs for enlarged collection from the periphery, with a total capacity of 730 μL. The design in FIG. 3c, instead, is characterized by shortened parallel channels in the central regions, and a tree structure fluidic network connected to the periphery. This configuration is preferable to the one in FIG. 3a if the sample collection more likely occurs in the central area of the pad. Since the flow cannot be directed, a drop that is collected from the center of a channel diffuses towards the reservoir, but also in the opposite direction towards the periphery, partially wasting the sample volume. Shortening the channels minimized this counter effect, but reduces the covered area. Consequently, a tree-like structure is inserted in the layout in order to collect sample also from the outer regions, but with a less convenient fluidic network. Finally, in the version in FIG. 3d, the channels are exactly the same as in the first design, but are not connected together in the central square reservoirs. This design is undoubtedly favorable since once the liquid sample reaches the central area it can only be absorbed by the layer underneath, i.e. the filtering membranes at the chip inlets, and is not flowing the opposite side of the collection pad. Designs 3c) and 3d) have volume capacity comparable to 3a), i.e. 620 μL. All the aforementioned designs, 3a) to 3d), have a final channel dimension on 1 mm. A fifth version (FIG. 3e) is characterized by central square reservoirs and parallel straight channels, similarly to 3a), but with channel width reduced to 500 μm. In this case the volume capacity is decreased to 400 μL. The lower is the volume capacity of the paper collection layer, the larger is the amount of gathered sample which will be absorbed by the filtering membranes and ultimately dragged inside the capillary chip.

Paper Collection Pad Trials with Clinical Samples

It is interesting to verify if the vaginal fluid absorbed by the collection pad and displaced by the paper fluidics is producing congruent result when subjected to immunoassay test, if compared with the direct testing of sample. Through a collaboration with the Centre Hospitalier Universitaire Vaudois, clinical samples are available to carry out this study. The hospital adopts the QuikCheck fFN™ Test Kit (Hologic, Inc.) for confirming the risk of preterm delivery, based on fetal Fibronectin biomarker. The vaginal fluid sample is collected through a swab and diluted in the dedicated buffer available in the immunoassay kit. Successively a dipstick for later flow assay is dipped in the sample buffered solution. If the protein is present in the fluid with a concentration above 50 ng*mL⁻¹, its binding with the complementary antibody generates a colored line on the stripe and the test is considered as positive. A second line, the control line, should appear confirming that the sample was correctly absorbed by the paper dipstick. If only the control line is appearing the test result is negative, while if no line is visible the test is not faithful.

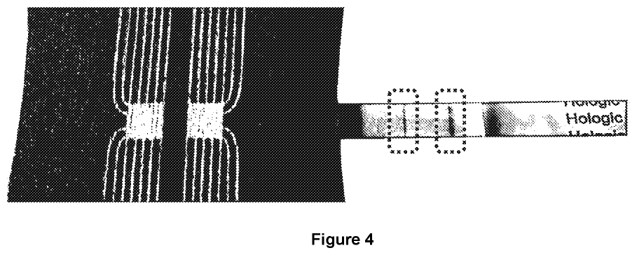

The same colorimetric assay can be performed attaching the absorbent part of the lateral flow assay to the reservoirs of the paper collection pad, as showed in FIG. 4. In this configuration, the sample is dispensed on the pad and dragged to the assay strip: if the sample is not altered the test should give the same result as the one obtained with the standard procedure. After accessing the fetal Fibronectin concentration with the test kit, the volume of buffered sample remaining after the analysis can be employed for performing the same test embedded in the absorbent collection pad of the invention. In particular two samples with 400 μL volume are tested; one resulted positive and one negative. The clinical samples are dispensed randomly over the collection pad surface. The selected design is the one with reduced channels width, showed in FIG. 3e. This latter is the most suitable for trials since, due to its low volume capacity, it retains less sample and enough liquid can be dragged to the lateral flow assay strip. Concerning the positive sample, two lines are visible on the test strip, while the negative sample only generates one. This confirms that the collection system does not affect the original result.

Real-Time Flow Monitoring Through Impedimetric Read-Out

In the implemented setting, six different sensing sites along the microfluidic path are introduced in the layout. Each site consists in a pair of golden planar electrodes. Measuring the impedance between the two electrodes it is possible to determine the presence of liquid at that particular position inside the chip. In particular, the sensing sites are distributed along the microfluidic path in correspondence of the inlet, the first resistor, the second resistor, the reaction chamber, the pump inlet and the pump outlet.

The core element for retrieving the impedance values is the AD5934 IC component (Analog Devices). The component AD5934 both provides the sinusoidal excitation voltage and collects the current flowing through the unknown impedance. It subsequently calculates the real and imaginary parts of the impedance under measurement, and stores them as digital 8-bit words. The expected liquid impedance to be measured in the microfluidic devices is compatible with the component specifications. Since on the capillary microfluidic chip twelve sensing sites are to be monitored, in order to employ only one impedance analyzer, the excitation voltage is multiplexed between the different measuring electrodes. In the chip metal layer layout, the pair of electrodes are intentionally designed having each measurement electrodes connected to a specific external pad, whereas the reference electrodes are all tied to the same pad. In this way, the output current to be collected from each sensing site and sent back to the AD5934 is retrieved from a unique pad, therefore avoiding the necessity of demultiplexing the output.

The microcontroller MCU, Bluno nano (DFRobot), is an Arduino programmable board. The microcontroller unit is in communication with the impedance analyzer through an 120 serial interface. 120, Inter-Integrated Circuit, is a serial protocol for a two-wire connection: SCL, standard clock signal, for timing synchronization and SDA, serial data, for data sharing. Through this serial bus interface and following the read/write specifications of the AD5934, it is possible to set the measurement parameters and to read the real and imaginary parts of the impedance from the device registers at each frequency in the programmed sweep. Furthermore, the microcontroller is managing the distribution of the excitation signal to the different sensing sites through the multiplexer. The MCU is connected to the four selector signals of the MUX: a specific combination of these signals is uniquely selecting one of the 12 channels of the MUX. The multiplexer enable signal is tied to ground, i.e. it is always active. The electronic circuit can be fabricated as printed circuit board with integrated components.

The microcontroller, Bluno nano, can be programmed in Arduino language and the scripts, named sketches, can be compiled and flashed to the board through the Arduino IDE software. The script contains the commands to be issued to the MUX for selecting one sensing site at a time and the ones for obtaining the measurement of impedance from the AD5934 IC component.

The AD5934 component is designed to perform impedance spectroscopy though a user-defined frequency sweep. To detect the presence of liquid it is enough to measure the impedance at a single frequency, e.g. 10 kHz; however, it is advantageous to perform several measurements at the same frequency and to average them, to increase the system sensitivity. Ten measurements are performed with the same parameters at each of the twelve sensing sites and the values are printed in real-time to the serial monitor of the Arduino IDE, together with the computed average. This operation is performed for the twelve pairs of electrodes on the microfluidic chip. The microcontroller selects the sensing site through the multiplexer. After the last site is accessed a power-down command is issued to the AD5934 component. The obtained values, however, are then converted in actual impedance values, multiplying them by a scale factor which has to be previously defined through a calibration step on a known resistance.

### Additional or Alternative Embodiments

In one embodiment, the electronic system does not contain any battery. The electronics contained in the pad according to the invention is powered wirelessly by e.g. an NFC module-containing device, such as a smartphone, which may additionally receive, store, analyze and distribute the test data. The user can activate the system using a dedicated external software, such as a smartphone app, for a suitable time lapse before wearing the pad. The test result can be read by the receiving device (e.g. smartphone) and sent to e.g. a user's doctor for deciding on further medical steps once the pad is unworn. The MCU can be configured to perform calibrations, test readings and communication through the NFC antenna.

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments, and be given the broadest reasonable interpretation in accordance with the language of the appended claims.

1-13. (canceled)

14. A vaginal fluid monitoring device embedded into a feminine sanitary pad, the device comprising:
an absorbent layer configured to be in proximity to, and collect, a vaginal fluid; and
a biosensing system in fluidic connection with the absorbent layer, the biosensing system including,
a microfluidic chip configured to perform an immunoassay to detect a presence and/or a concentration of a target biomarker comprised in a vaginal fluid, the target biomarker indicative of a pre-term birth (PTB) risk and/or premature rupture of membrane (PROM) risk,
a readout device for providing a readout of the presence and/or the concentration of the target biomarker, and
an electrode array located along the microfluidic chip configured to detect and analyze a flow of the vaginal fluid by an impedance device.

15. The monitoring device of claim 14, wherein the target biomarker is selected from a group comprising Foetal Fibronectin (fFn), Insulin-like growth factor-binding protein 1 (IGFBP-1), Placental alpha microglobulin-1 (PAMG-1), Inflammatory cytokines including IL-1A, IL1B, IL-2, IL-6 and IL-8, Tumor necrosis factor-alpha (TNF-alpha), C-reactive protein (CRP), Alpha-Fetoprotein (AFP) and Corticotropin-releasing hormone (CRH), cystatin A (CSTA), monocyte/neutrophil elastase inhibitor (SERPINB1), squamous cell carcinoma antigen 1 (SERPINB3), squamous cell carcinoma antigen 2 (SERPINB4), interleukin-1 receptor antagonist (IL1RN), thioredoxin-1 (TXN), Zn-superoxide dismutase (SOD1), peroxiredoxin-2 (PRDX2), and glutathione S-transferase pi (GSTP1), epidermal fatty acid binding protein 5 (FABP5), annexin A3 (ANXA3), albumin (ALB), cysteine protease (CSTA), matrix metalloproteinases (MMPV, TIMP2 & TIMP1), Vitamin D binding protein (GC, group-specific component), a-fetoprotein, major basic protein, placental isoferritin, corticotropin-releasing hormone, adrenocorticotropin, prolactin, human chorionic gonadotropin, C-terminal propeptide of procollagen, sialidase, municase, as well as antigens derived from herpes virus, vaginal gonococcus, chlamydia, group beta streptococcus, polymorphonuclear leukocytes and clue cells.

16. The monitoring device of claim 14, wherein the readout device for providing the readout includes an optical readout and/or an electrochemical readout.

17. The monitoring device of claim 16, wherein the readout device for providing the optical readout includes an optical detector for detecting an optical signal generated by the immunoassay.

18. The monitoring device of claim 17, wherein the optical detector comprises a laser light source coupled with a photodiode.

19. The monitoring device of claim 14, wherein the absorbent layer is configured to keep a constant flow of a vaginal fluid inside the microfluidic chip.

20. The monitoring device of claim 14, wherein the microfluidic chip is configured to operate in a passive capillary regime.

21. The monitoring device of claim 14, wherein the absorbent layer includes a substrate patterned with fluidic paths.

22. The monitoring device of claim 14, further comprising:
a microprocessor configured for processing a signal generated by the biosensing system.

23. The monitoring device of claim 22, wherein the microprocessor is configured for processing a signal selected from an electrical impedance signal and/or an optical signal.

24. The monitoring device of claim 14, wherein the absorbent layer and the biosensing system are arranged as layers of a stack.

25. A method for detecting a target biomarker in a vaginal fluid, the method comprising the steps of:
allowing a vaginal fluid monitoring device to collect a vaginal fluid, the vaginal fluid monitoring device including,
an absorbent layer configured to be in proximity to, and collect, a vaginal fluid, and
a biosensing system in fluidic connection with the absorbent layer, the biosensing system including,
a microfluidic chip configured to perform an immunoassay to detect a presence and/or a concentration of the target biomarker in the vaginal fluid,
a readout device for providing a readout of the presence and/or the concentration of the target biomarker, and
an electrode array located along the microfluidic chip configured to detect and analyze a flow of the vaginal fluid by an impedance device; and
obtaining the readout of the presence and/or the concentration of the target biomarker to determine the presence and/or the concentration of the target biomarker in the vaginal fluid.

26. The method of claim 25, wherein the presence and/or the concentration of the target biomarker is indicative of a pre-term birth (PTB) risk and/or premature rupture of membrane (PROM) risk.

27. The method of claim 25, wherein the target biomarker is selected from a group comprising Foetal Fibronectin (fFn), Insulin-like growth factor-binding protein 1 (IGFBP-1), Placental alpha microglobulin-1 (PAMG-1), Inflammatory cytokines including IL-1A, IL1B, IL-2, IL-6 and IL-8, Tumor necrosis factor-alpha (TNF-alpha), C-reactive protein (CRP), Alpha-Fetoprotein (AFP) and Corticotropin-releasing hormone (CRH), cystatin A (CSTA), monocyte/neutrophil elastase inhibitor (SERPINB1), squamous cell carcinoma antigen 1 (SERPINB3), squamous cell carcinoma antigen 2 (SERPINB4), interleukin-1 receptor antagonist (IL1RN), thioredoxin-1 (TXN), Zn-superoxide dismutase (SOD1), peroxiredoxin-2 (PRDX2), and glutathione S-transferase pi (GSTP1), epidermal fatty acid binding protein 5 (FABP5), annexin A3 (ANXA3), albumin (ALB), cysteine protease (CSTA), matrix metalloproteinases (MMPV, TIMP2 & TIMP1), Vitamin D binding protein (GC, group-specific component), a-fetoprotein, major basic protein, placentalisoferritin, corticotropin-releasing hormone, adrenocorti-cotropin, prolactin, human chorionic gonadotropin, C-terminal propeptide of procollagen, sialidase, municase, as well as antigens derived from herpes virus, vaginal gonococcus, chlamydia, group beta streptococcus, polymorphonuclear leukocytes and clue cells.
